(19) United States
(12) Patent Application Publication
(10) Pub. No.: US 2016/0228290 A1
(43) Pub. Date: Aug. 11, 2016

(54) Personal Cooling Device

(71) Applicant: EUMAR TECHNOLOGY LIMITED, Whitestone, Hereford (GB)

(72) Inventor: Euan Eric Neil Thomas George Davidson, Hampshire (GB)

(21) Appl. No.: 15/021,819

(22) PCT Filed: Sep. 12, 2014
(86) PCT No.: PCT/GB2014/052783

§ 371 Date: Mar. 14, 2016

(30) Foreign Application Priority Data
Sep. 13, 2013 (GB) 1316325.8
Nov. 11, 2013 (GB) 1319831.2

(51) Int. Cl. A61F 7/10; A61F 7/02

(52) U.S. Cl. CPC A61F 7/10; A61F 7/02; A61F 2007/0036

(57) ABSTRACT

The present invention relates to a personal cooling device, in particular a device incorporating ice. There is provided a personal cooling device comprising an envelope at least partially formed of partially liquid permeable material surrounding an absorbent material, the envelope having a sealable opening into which ice or another frozen material can be inserted. The absorbency of the absorbent material being sufficient to absorb the majority of water with only a small proportion of the water passing though the permeable material.

The present invention relates to a personal cooling device, in particular a device incorporating ice.

In hot weather or in hot places it can be pleasant to hold something cool against the body. It is known to place ice within a towel and hold it against the head or body. However, as the ice melts, the absorbency of the towel is soon overwhelmed and water leaks out from the towel. While the provision of a small amount of water is pleasant, as its evaporation from the skin provides an additional cooling effect, excessive water can be uncomfortable.

The object of the present invention is to provide an improved personal cooling device.

According to the invention there is provided a personal cooling device comprising:
absorbent material;
an envelope at least partially formed of partially liquid permeable material around the absorbent material, the envelope being sealed around a substantial portion of its perimeter leaving an opening into which ice or another frozen material can be inserted;
means for closing the envelope at the opening.

The invention is such that in use ice or another frozen material can be inserted into the envelope and the envelope sealed, the absorbency of the absorbent material being sufficient to absorb the majority of water created by melting of the ice or by condensation with a small proportion of the water passing though the at least partially liquid permeable material for providing additional cooling for the user.

The personal cooling device can then be used to cool a body down by pressing against part of the body, for example the temples or back of the neck. The small proportion of the water that is not absorbed passing out of the device to be evaporated by the body for additional cooling. With most of the water being absorbed, the water does not create a nuisance.

The envelope maybe formed entirely of partially liquid permeable material or maybe of partially liquid permeable material on one layer and of impermeable material on another layer.

Preferably the absorbent material is sealed within a wall of the envelope. The absorbent material maybe sealed between partially liquid permeable material layers or maybe sealed between a partially liquid permeable material layer and an impermeable material layer.

Usually the absorbent material will incorporate super-absorbent material, in powder form, in fibre form, or in both forms. However, the absorbent material may additionally or alternatively include fluff pulp, or any other type of absorbent material.

In one embodiment the envelope comprises:
a first piece of at least partially liquid permeable material;
absorbent material;
a second piece of at least partially liquid permeable material,

the permeable material forming the envelope around the absorbent material, the envelope being sealed around a substantial portion of its perimeter leaving an opening into which ice or another frozen material can be inserted.

Preferably the absorbent material will be sealed to at least one piece of the permeable material. Usually the absorbent material will be in the form of a fabric.

Conveniently the absorbent material may be provided within the envelope sealed beneath a further piece of at least partially liquid permeable material.

In another embodiment the envelope comprises:
a first piece of at least partially liquid permeable material;
absorbent material;
a second piece of at least partially liquid permeable material; the first and second pieces of at least partially liquid permeable material being sealed together capturing the absorbent material therebetween, creating an absorbent pad;
a third piece of at least partially liquid permeable material, the third piece being sealed to the absorbent pad around a substantial portion of their perimeters, creating the envelope having an opening.

Usually the means for sealing the envelope will be a pressure adhesive. Typically this will be provided under a release tape. Alternatively it could be a hook and loop fastening, or any other similar sealing material.

In some embodiments the device may further include a hand strap, for holding the device against a user's hand. The hand strap may be a layer of stretchable material, which is attached across the device. A user can place his hand between the strap and the device to hold the device to his hand. Alternatively the hand strap may be of the same material as the pieces of at least partially liquid permeable material.

Usually the permeable material will be a non-woven air laid material. However, some or all of the material may be a one-way perforated material, typically polyethylene. Such material is provided with shaped micro-perforations that enable fluid to flow through the material in one direction, but discourage flow in the opposite direction. The material can be positioned with the apertures directed to reduce flow of water out of the device. This can be used to provide additional control to the amount of water able to pass out of the device. Further reducing the flow of melt water out of the device.

Where super-absorbent material is provided, usually it will be incorporated into other absorbent material, typically cellulose. Alternatively the absorbent material may be a starch based absorbent material. Such a material is biodegradable.

Additionally, the permeable material could also be made of starch-based materials, or other biodegradable plastics materials.

In a further alternative the permeable material, or at least the permeable material surrounding the opening of the envelope, can be coated in aluminium or other reflective material, to reflect heat and reduce the rate of ice melting.

To help understanding of the invention, a specific embodiment thereof will now be described by way of example and with reference to the accompanying drawings, in which:

FIG. 1 is a top view of a personal cooling device according to the invention;

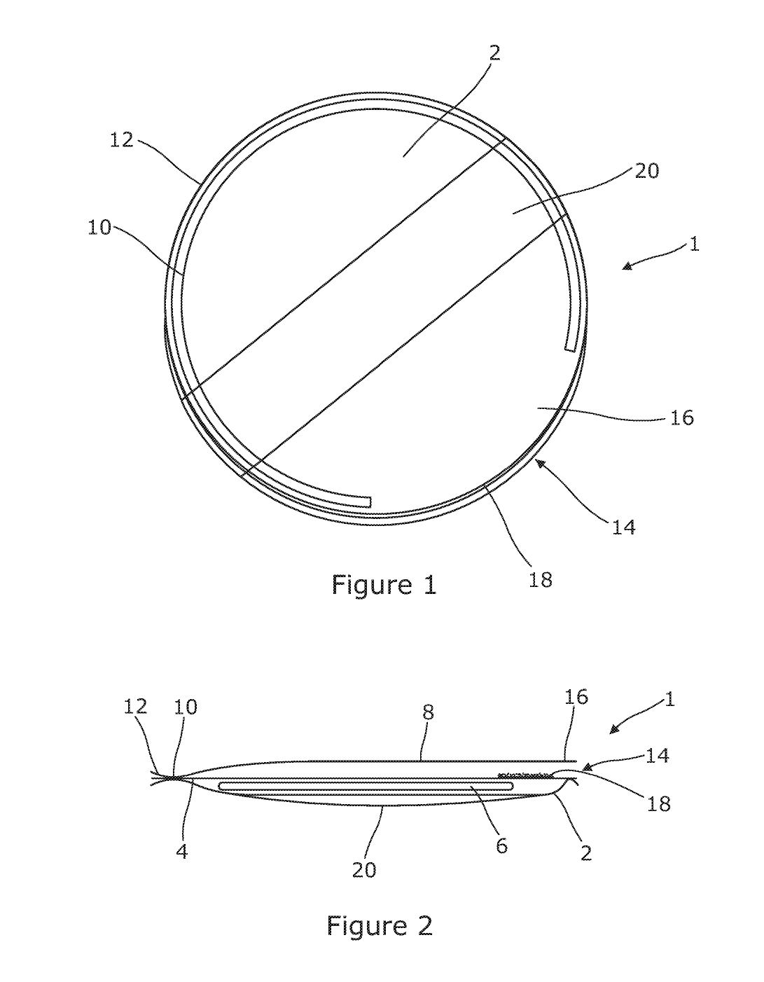

FIG. 2 is a cross-sectional view of the device of FIG. 1;

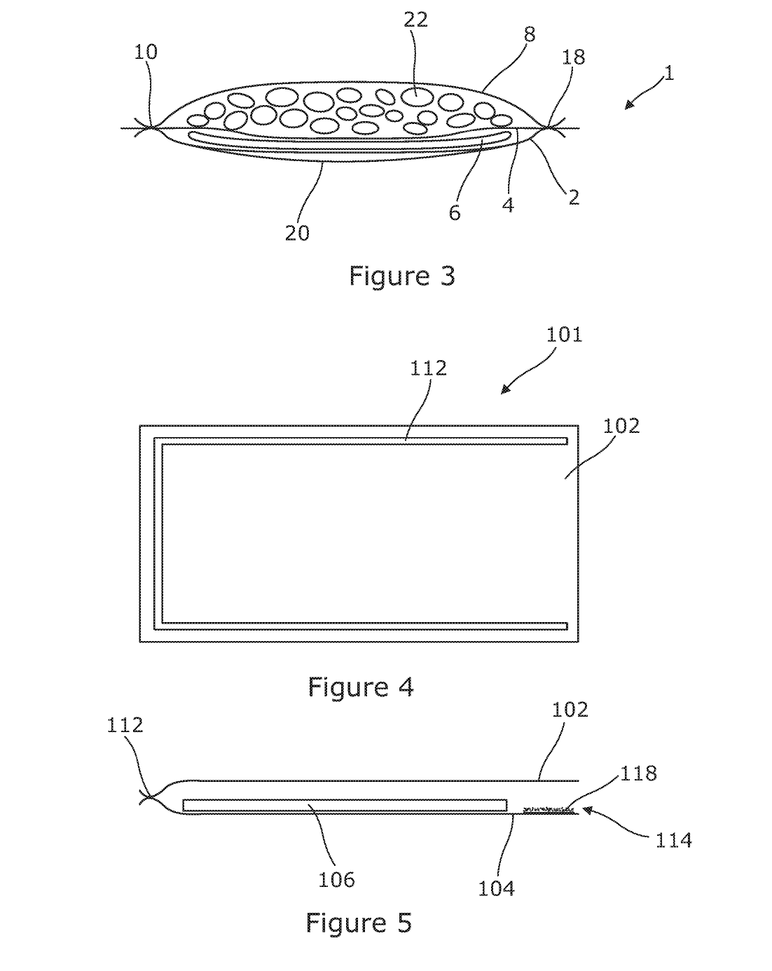

FIG. 3 is a cross-sectional view of the device of FIG. 1 filled with ice and sealed for use;

FIG. 4 is a top view of a second embodiment of a personal cooling device according to the invention;

FIG. 5 is a cross-sectional view of the device of FIG. 4; and

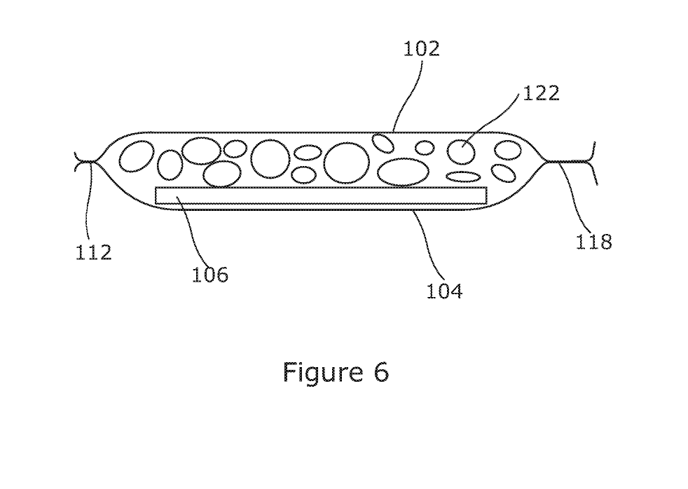

FIG. 6 is a cross-sectional view of the device of FIG. 4 filled with ice and sealed for use.

Referring to the drawings, the personal cooling device 1 comprises an outer permeable layer 2 and a second permeable layer 4, sealed to the outer permeable 2 around their perimeters. Sandwiched between the permeable layers is an absorbent layer 6, which is fully contained within the seal.

A third permeable layer 8 is provided over one of the permeable layers, as shown the second permeable layer 4. This is sealed 10 to the second permeable layer 4 around a substantial portion of its perimeter 12. As shown the device is substantially circular in shape, although it may be provide in any shape, typically it will be circular, oval, rectangular or square. As shown the seal 10 is provided over approximately three-quarters of the perimeter 12. This creates a pocket 14, between the second permeable layer 4 and the third permeable layer 8.

The unsealed portion 16 of the perimeter 12 is provided with means for closure 18, typically as shown a pressure adhesive covered by a release tape. However, hook and loop fastening or other sealing means could be used.

The permeable layers 2, 4, 8 may be made of non-woven, air laid material, of material including polyethylene coated polypropylene fibres. This allows the material to be sealed together by heat sealing. Alternatively the permeable layers may be made of any other at least partially liquid permeable material, including woven materials. They may be sealed together heat, glue, ultrasonic welding, sewing or any other attachment means. In a further alternative one or more of the permeable layers may be made of one way perforated polyethylene sheet, assembled to reduce liquid from escaping from the device. Such material is provided with shaped micro-perforations that enable fluid to flow through the material in one direction, but discourage flow in the opposite direction. The material can be positioned with the apertures directed to reduce flow of water out of the device.

The sides of the permeable layers 4, 8 adjacent the pocket 14 are coated with aluminium, or any other reflective material. This reduced heating of the ice, reducing the speed of melting of the ice. This is optional and will not be use used in all embodiments.

The device also includes a hand strap 20, although this is optional and will not be included in all embodiments. This is attached over the outer permeable layer 2, and is attached across the device 1 so that a user can insert his hand between the strap 20 and the device 1 to secure the device to his hand. The hand strap may be made of the same material as the permeable layers. Alternatively it may be made from a stretchable material for improved connection between the device and a user's hand. The stretchable material may also be a non-woven material.

For use, ice 22 is placed in the pocket 14, and the final portion 16 of the perimeter is sealed.

This creates a cooling pad for personal use. A user will insert his hand under the hand strap 20 and press the pad against his skin, for exmple temples, wrist or the back of his neck, providing a cooling to his skin.

As the ice 22 melts, the majority of the water produced will be absorbed by the absorbent layer 6. However, before contact with the absorbent layer, a small proportion of the water created by the melting ice will pass out of the pad through the permeable layers. This is advantageous as the evaporation of water from the skin provides an additional cooling effect for the user. In addition the thermal contact between the pad and the user is greatly enhanced by the presence of this water.

Usually the absorbent layer will include super-absorbent material that is able to hold many times its weight in water. In addition the super-absorbent material bonds with the water so that the water cannot be released. The super-absorbent material may be provided in powder form, in fibre form or in both forms. Usually it will be provided with additional absorbent material, typically cellulose, to form a sheet of absorbent material. It may also be combined with non-absorbent material to form a cohesive sheet. Alternatively the absorbent material may be a starch based absorbent material. Such a material is biodegradable. Alternatively the absorbent material may not include any super-absorbent material. For example fluff pulp, a type of paper or wood pulp may be used. In a further alternative, the absorbent material may be a mixture of super-absorbent material and other absorbent material.

Now turning to FIGS. 4 and 5, the device 101 thereshown includes a first permeable layer 102 and a second permeable layer 104 sandwiching therebetween an absorbent layer 106. The absorbent layer may be sealed or otherwise attached to one of the permeable layers or may be loose within the layers. The first and second permeable layers 102, 104, are sealed together around a substantial portion of their perimeter 112. This perimeter seal may include sealing to the absorbent layer. As shown the pad is in the shape of a square, and the two layers 102, 104 are sealed on three sides. However, as discussed above, the pad could be of any shape. The unsealed portion of the perimeter 112 is provided with a layer of adhesive covered by a release strip 118. This creates a pocket 114, in which is held the absorbent layer 106.

In use, the pocket 114 is filled with ice 122, and is sealed by removal of the release strip 118 and pressing the layers 102, 104 together.

A user can then hold the pad against his skin, providing a cool effect. As with the first embodiment, the absorbent layer 106 absorbs most of the water produced by the melting of the ice. However, as small proportion of the water leaks out of the device before it is absorbed by the absorbent layer. As before this is advantageous as it improves the thermal contact between the users skin and the device, and it provides moisture which can evaporate from the skin cooling the user.

The absorbent layer ensures that while some of the water produced by the melting of the ice passes out of the device, the majority of the water is absorbed. As a result the user does not have the difficulty of containing the full volume of melt water produced by the ice.

The volume and proportion of water retained by the pad and allowed to leak or pass out of the pad can be controlled by the amount of super-absorbent material provided in the pad, and by the use of one way perforated material. As set out above, this material may be used as some or all of the permeably layers. The micro-perforations are designed such that water is encouraged to flow through the material in one direction but discouraged from flowing in the other direction. This is due to the shape of the perforations, which are substantially conical.

If this material is used as the permeable layer between the users hand and the absorbent material it will permit very little water to pass out of the pad towards the users hand, as the water will have contacted the absorbent layer. However, if it is used between the ice and the users skin, it will allow a certain amount but very little water to pass out of the device.

In addition the layers adjacent the ice can be coated with aluminium or other reflective material, to slow down the melting of the ice. This will prolong the time before the ice melts in the device.

The invention is not intended to be restricted to the details of the above-described embodiment. For instance, pre-frozen, coolant containing pouches can be inserted into the pocket of the device. The absorbent layer will absorb condensation forming of the coolant pouches, and the permeable layers will separate the skin from the coolant pouch.

1-19. (canceled)

20. A personal cooling device comprising:
a closable envelope of at least partially liquid permeable material for accommodating ice or another frozen material, the envelope:
enclosing absorbent material sealed within a wall of the envelope, the absorbent material being sealed between a partially liquid permeable material layer and an impermeable material layer
being sealed around a substantial portion of its perimeter leaving an opening into which the ice or another frozen material can be inserted;
having means for closing the envelope at the opening.

21. The personal cooling device according to claim 20, wherein the envelope is entirely of partially liquid permeable material.

22. The personal cooling device according to claim 20, wherein the envelope is of partially liquid permeable material on one layer and of impermeable material on another layer.

23. The personal cooling device according to claim 20, wherein the means for sealing the envelope is a pressure adhesive.

24. The personal cooling device according to claim 20, wherein the means for closing the envelope is a release tape, hook and loop fastening, or any other similar sealing material.

25. The personal cooling device according to claim 20, wherein the absorbent material is combined with non-absorbent material.

26. The personal cooling device according to claim 20, wherein the absorbent material is super-absorbent material, fluff pulp and/or any other type of absorbent material.

27. The personal cooling device according to claim 21, wherein the super-absorbent material is in powder and/or fibre form.

28. The personal cooling device according to claim 20, wherein the permeable material is air laid non-woven material.

29. The personal cooling device according to claim 20, wherein some or all of the permeable material is a one-way perforated material that enables fluid to flow through the material in predominately one direction.

30. The personal cooling device according to claim 29, wherein the permeable material is polyethylene.

31. The personal cooling device according to claim 20, wherein at least the permeable material surrounding the opening of the envelope is coated in a reflective material, to reflect heat and reduce the rate of ice melting.

32. The personal cooling device according to claim 20, wherein the ice or another frozen material further comprises an additive that is beneficial to the user's skin and/or enhances the cooling effect of the device.

33. The personal cooling device according to claim 20, wherein the additive is aloe vera.

34. The personal cooling device according to claim 20, wherein the device further includes a hand strap for holding the device against a user's hand.

35. The personal cooling device according to claim 34, wherein the hand strap is formed of a layer of stretchable material and/or of at least partially liquid permeable material.
